(19) United States
(12) Patent Application Publication
(10) Pub. No.: US 2026/0144448 A1
(43) Pub. Date: May 28, 2026

(54) BLOOD PRESSURE MEASUREMENT DEVICE, BLOOD PRESSURE MEASUREMENT METHOD, AND BLOOD PRESSURE MEASUREMENT RECORDING MEDIUM

(71) Applicant: OMRON HEALTHCARE Co., Ltd., Kyoto (JP)

(72) Inventors: Daizo OKA, Kyoto (JP); Yuki OTA, Kyoto (JP); Wataru TSUNODA, Kyoto (JP); Naomi MATSUMURA, Kyoto (JP)

(21) Appl. No.: 19/453,570

(22) Filed: Jan. 20, 2026

Related U.S. Application Data
(63) Continuation of application No. PCT/JP2024/040873, filed on Nov. 18, 2024.

(30) Foreign Application Priority Data
Feb. 27, 2024 (JP) 2024-027771

(51) Int. Cl. A61B 5/0225; A61B 5/0235

(52) U.S. Cl. CPC A61B 5/0225; A61B 5/0235; A61B 2562/0247

(57) ABSTRACT

A blood pressure measurement device includes a cuff to be wrapped around a measurement target site, a pressure detection unit configured to detect a cuff pressure, a pressure control unit configured to control the cuff pressure, a blood pressure calculation unit that calculates based on the cuff pressure, and a wrapping strength determination unit configured to determine a wrapping strength of the cuff, in which the pressure control unit performs a constant-pressure control to maintain the cuff pressure at a constant target pressure value from a start of an increase in the cuff pressure to an end of a wrapping strength determination processing, and performs a constant-speed pressurization control to increase the cuff pressure at a constant speed for calculating a blood pressure after the end of the wrapping strength determination processing, and the wrapping strength determination unit determines the wrapping strength during the constant-pressure control.

### CROSS-REFERENCE TO RELATED APPLICATIONS

This application is the U.S. national stage application filed pursuant to 35 U.S.C. 365(c) and 120 as a continuation of International Patent Application No. PCT/JP2024/040873, filed Nov. 18, 2024, which application claims priority to Japanese Patent Application No. 2024-027771, filed Feb. 27, 2024, which applications are incorporated herein by reference in their entireties.

### TECHNICAL FIELD

The present invention relates to a blood pressure measurement device, a blood pressure measurement method, and a blood pressure measurement program.

### BACKGROUND ART

In recent years, it has become widespread to perform health management by measuring information related to a body and health of an individual, such as a blood pressure value, by using a measurement apparatus, and recording and analyzing the measurement result. As an example of the measurement apparatus as described above, a sphygmomanometer is known which has a function of determining a wrapping strength of a cuff worn on a measurement target site such as an upper arm or a wrist of a subject(see Patent Document 1).

In the sphygmomanometer disclosed in Patent Document 1, after the cuff is worn, a pump is driven by applying a constant voltage to the pump during initial pressurization, the wrapping strength of the cuff is determined from a cuff pressure-pressurization time characteristic (cuff pressure curve) from an initial pressure to a pressure value at the end of the initial pressurization, and subsequently, blood pressure calculation processing is performed.

However, in such constant-voltage driving, the initial pressurization level cannot be set too low in order to secure a time required for the cuff wrapping strength. In addition, since it is necessary to acquire a pressure pulse wave for blood pressure calculation immediately after the cuff wrapping strength determination, noise generated at the timing of control switching may adversely affect the pressure pulse wave.

### CITATION LIST

Patent Literature 1: JP 5408142 B

### SUMMARY OF INVENTION

### Technical Problem

In view of the related art as described above, an object of the present invention is to provide a technique capable of suppressing the influence of noise at the timing of control switching from cuff wrapping strength determination to blood pressure calculation.

### Solution to Problem

In order to solve the above problems, the present invention provides a blood pressure measurement device including: a cuff to be wrapped around a measurement target site; a pressure detection unit configured to detect a cuff pressure in the cuff; a pressure control unit configured to control the cuff pressure; a blood pressure calculation unit configured to perform blood pressure calculation processing to calculate a blood pressure of a subject on the basis of the cuff pressure detected; and a wrapping strength determination unit configured to perform wrapping strength determination processing to determine a wrapping strength of the cuff to the measurement target site, in which the pressure control unit performs a constant-pressure control to maintain the cuff pressure at a constant target pressure value from a start of an increase in the cuff pressure to an end of the wrapping strength determination processing, and performs a constant-speed pressurization control to increase the cuff pressure at a constant speed for calculating the blood pressure after the end of the wrapping strength determination processing, and the wrapping strength determination unit determines the wrapping strength during the constant-pressure control.

According to the above configuration, in the constant-pressure control for cuff wrapping strength determination, the control is switched from a state in which the cuff pressure is maintained at a constant target pressure value to the constant-speed pressurization control for blood pressure calculation, thereby making it possible to suppress the influence of noise on blood pressure calculation at the timing of switching.

In the present invention, the wrapping strength determination unit may determine the wrapping strength on the basis of a change in the cuff pressure during a determination target period during which the cuff pressure reaches the target pressure value from an initial pressure, and stabilizes.

According to the above configuration, the wrapping strength can be determined according to the magnitude of a pressure applied to the measurement target site by the cuff pressure when the cuff is wrapped around the measurement target site.

In the present invention, the wrapping strength determination unit may determine the wrapping strength by comparing the number of times the cuff pressure becomes less than the target pressure value during the determination target period with a predetermined threshold, and comparing a difference from the target pressure value with a predetermined threshold.

According to the above configuration, it is possible to determine, as the wrapping strength, whether the magnitude of a pressure applied to the measurement target site by the cuff pressure when the cuff is wrapped around the measurement target site is smaller than an appropriate value.

In the present invention, the wrapping strength determination unit may determine the wrapping strength on the basis of whether the cuff pressure increases or decreases relative to the target pressure value during the determination target period.

According to the above configuration, it is possible to determine, as the wrapping strength, whether the magnitude of a pressure applied to the measurement target site by the cuff pressure when the cuff is wrapped around the measurement target site is greater than an appropriate value.

In the present invention, the target pressure value may be smaller than 30 mmHg.

According to the above configuration, since the constant-pressure control is performed using the cuff pressure smaller than 30 mmHg as the target pressure value, the constant-speed pressurization control for blood pressure calculation can be started from a state in which the cuff pressure is lower.

In the present invention, the pressure control unit may perform the constant-speed pressurization control depending on a result of the wrapping strength determination processing.

According to the above configuration, when the wrapping strength is appropriate, the constant-speed pressurization control is performed, thereby making it possible to realize high-accuracy blood pressure calculation. In addition, when the wrapping strength is not appropriate, the air in the cuff is exhausted without performing the constant-speed pressurization control, thereby making it possible to reduce the burden on the subject.

In the present invention, the pressure control unit may perform the constant-pressure control by a PID control.

In the present invention, the pressure control unit may control a pump configured to supply air to the cuff, and an exhaust valve configured to control exhaust from the cuff.

The present invention provides a blood pressure measurement method including: detecting a cuff pressure in a cuff wrapped around a measurement target site; performing a constant-pressure control to maintain the cuff pressure at a constant target pressure value; performing wrapping strength determination processing to determine a wrapping strength of the cuff to the measurement target site during the constant-pressure control; performing a constant-speed pressurization control to increase the cuff pressure at a constant speed after an end of the wrapping strength determination processing; and performing blood pressure calculation processing to calculate a blood pressure of a subject on the basis of the cuff pressure during the constant-speed pressurization control.

According to the above configuration, in the constant-pressure control for cuff wrapping strength determination, the control is switched from a state in which the cuff pressure is maintained at a constant target pressure value to the constant-speed pressurization control for blood pressure calculation, thereby making it possible to suppress the influence of noise on blood pressure calculation at the timing of switching.

The present invention provides a blood pressure measurement recording medium (or program) configured to cause a computer to execute: detecting a cuff pressure in a cuff wrapped around a measurement target site; performing a constant-pressure control to maintain the cuff pressure at a constant target pressure value; performing wrapping strength determination processing to determine a wrapping strength of the cuff to the measurement target site during the constant-pressure control; performing a constant-speed pressurization control to increase the cuff pressure at a constant speed after an end of the wrapping strength determination processing; and performing blood pressure calculation processing to calculate a blood pressure of a subject on the basis of the cuff pressure during the constant-speed pressurization control.

According to the above configuration, in the constant-pressure control for cuff wrapping strength determination, the control is switched from a state in which the cuff pressure is maintained at a constant target pressure value to the constant-speed pressurization control for blood pressure calculation, thereby making it possible to suppress the influence of noise on blood pressure calculation at the timing of switching.

### Advantageous Effects of Invention

According to the present invention, the influence of noise at the timing of control switching from cuff wrapping strength determination to blood pressure calculation can be suppressed.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic diagram illustrating a hardware configuration of a blood pressure measurement device according to Example 1;

FIG. 2 is a functional block diagram of the blood pressure measurement device according to Example 1;

FIG. 3 is a flowchart showing an entire processing procedure of the blood pressure measurement device according to Example 1;

FIG. 4 is a graph illustrating temporal changes in a cuff pressure and a pressure pulse wave during use of the blood pressure measurement device according to Example 1;

FIG. 5 is a flowchart showing a procedure of wrapping strength determination processing in the blood pressure measurement device according to Example 1;

FIG. 6 is a graph illustrating variation patterns of the cuff pressure for each wrapping strength in the blood pressure measurement device according to Example 1; and,

FIG. 7 is a schematic diagram illustrating a hardware configuration of a blood pressure measurement device according to Example 2.

### DESCRIPTION OF EMBODIMENTS

It is understood that this disclosure is not limited to the particular methodology, materials and modifications described and as such may, of course, vary. It is also understood that the terminology used herein is for the purpose of describing particular aspects only, and is not intended to limit the scope of the claims. Those in the art will understand that any suitable material, now known, or hereafter developed, may be used in forming the present invention described herein.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments, or it may be excluded.

Embodiments of the present invention will be specifically described below with reference to the drawings.

### Example 1

Hereinafter, an example of the embodiments of the present invention will be described. It should be noted that the dimension, material, shape, relative arrangement, and the like of the components described in the present examples are not intended to limit the scope of this invention to them alone, unless otherwise stated.

### Device Configuration

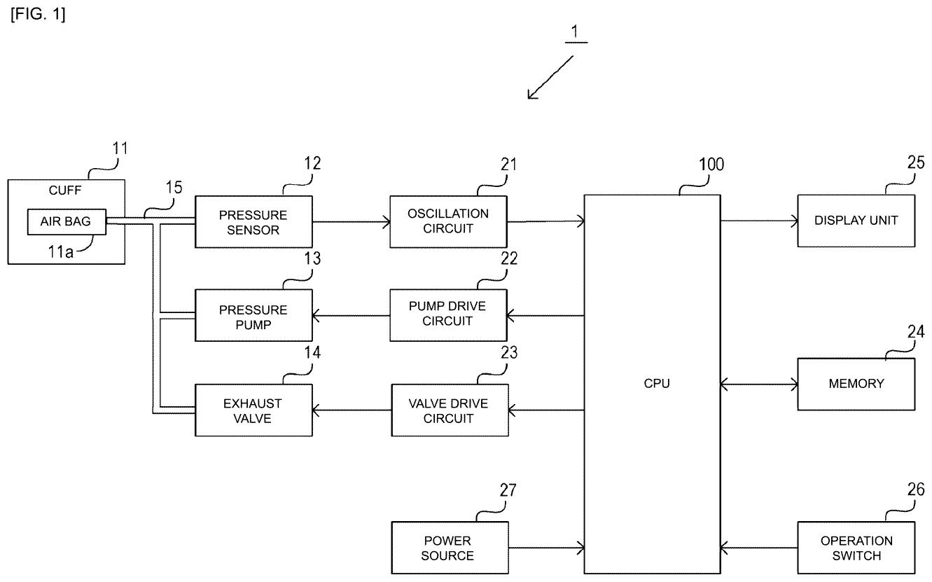

FIG. 1 is a schematic diagram illustrating a hardware configuration of a blood pressure measurement device 1 according to Example 1.

The blood pressure measurement device 1 includes a cuff 11, a pressure sensor 12, a pressure pump 13, an exhaust valve 14, an air tube 15, an oscillation circuit 21, a pump drive circuit 22, a valve drive circuit 23, a display unit 25, a memory 24, an operation switch 26, a power source 27, and a CPU 100. The blood pressure measurement device 1 corresponds to a blood pressure measurement device of the present invention. The cuff 11, the pressure pump 13, and the exhaust valve 14 correspond to a cuff, a pump, and an exhaust valve of the present invention, respectively.

The cuff 11 includes an air bag 11a in which air is contained. The cuff 11 is provided with the pressure sensor 12 for detecting a pressure in the air bag 11a of the cuff 11 (hereinafter, referred to as “cuff pressure”), the pressure pump 13 for supplying air to the air bag 11a, and the exhaust valve 14 that is opened and closed for maintaining the pressure in the air bag 11a or discharging the air in the air bag 11a, via the air tube 15.

The blood pressure measurement device 1 further includes the central processing unit (CPU) 100 for controlling each unit of the device, the memory 24 for storing a program that is executed for wrapping strength determination processing and blood pressure measurement processing described below and data such as a blood pressure measurement result, the display unit 25 for displaying various types of information such as a wrapping strength determination result and a blood pressure measurement result, the operation switch 26 for inputting various instructions for measurement, and the power source 27 for supplying power to each unit of the device, such as the CPU.

In addition, the oscillation circuit 21 outputs to the CPU 100 a signal having an oscillation frequency corresponding to an output value of the pressure sensor 12. The pump drive circuit 22 controls driving of the pressure pump 13, based on a control signal output from the CPU 100. The valve drive circuit 23 controls the opening and closing of the exhaust valve 14, based on a control signal output from the CPU 100.

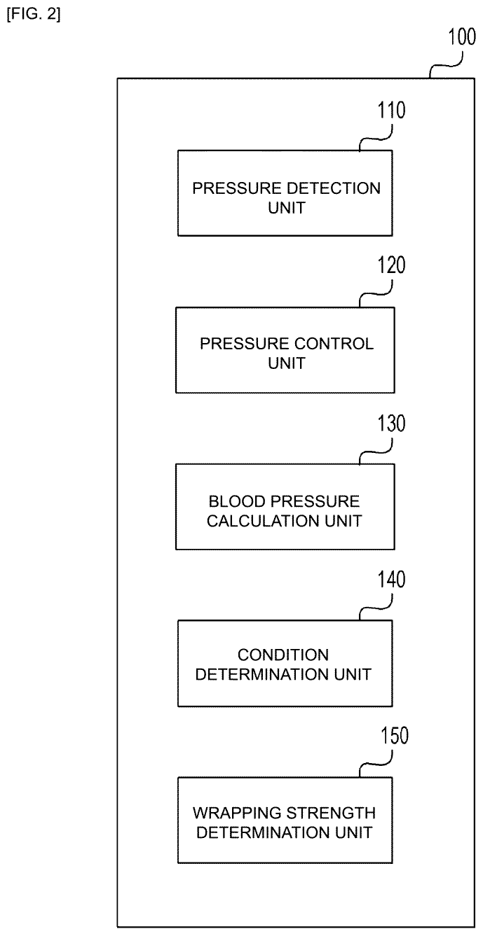

FIG. 2 is a functional block diagram of the blood pressure measurement device 1.

The CPU 100 includes a pressure detection unit 110, a pressure control unit 120, a blood pressure calculation unit 130, a condition determination unit 140, and a wrapping strength determination unit 150. The pressure detection unit 110, the pressure control unit 120, the blood pressure calculation unit 130, and the wrapping strength determination unit 150 correspond to a pressure detection unit, a pressure control unit, a blood pressure calculation unit, and a wrapping strength determination unit of the present invention, respectively.

An output signal of the oscillation circuit 21 is input to the pressure detection unit 110. The pressure detection unit 110 detects an oscillation frequency of the input signal and converts the detected oscillation frequency into a pressure value signal. The pressure detection unit 110 includes an HPF (High Pass Filter) unit that extracts and outputs a pressure pulse wave signal by performing HPF processing on the pressure value signal, and an LPF (Low Pass Filter) unit that extracts and outputs a pressure absolute value signal by performing LPF processing on the pressure value signal.

The pressure control unit 120 controls a cuff pressure of the cuff 11 by controlling operations of the pump drive circuit 22 and the valve drive circuit 23.

The blood pressure calculation unit 130 receives the pressure pulse wave signal extracted by the HPF unit of the pressure detection unit 110, and processes the received pressure pulse wave signal in accordance with an oscillometric method, thereby calculating a diastolic blood pressure (the lowest blood pressure) and a systolic blood pressure (the highest blood pressure).

A cuff pressure signal indicating the cuff pressure detected in a time-series manner is input from the LPF unit of the pressure detection unit 110 to the condition determination unit 140. The condition determination unit 141 determines whether a condition (determination condition) for performing wrapping strength determination is satisfied, based on the cuff pressure signal input in a time-series manner.

The cuff pressure signal indicating the cuff pressure detected in a time-series manner is input from the LPF unit of the pressure detection unit 110 to the wrapping strength determination unit 150, and the input cuff pressure signal is stored in a predetermined area of the memory 24. The wrapping strength determination unit 150 waits until the determination condition is satisfied, and then performs wrapping strength determination described below based on the cuff pressure signal read from the memory 24.

### Blood Pressure Measurement Method

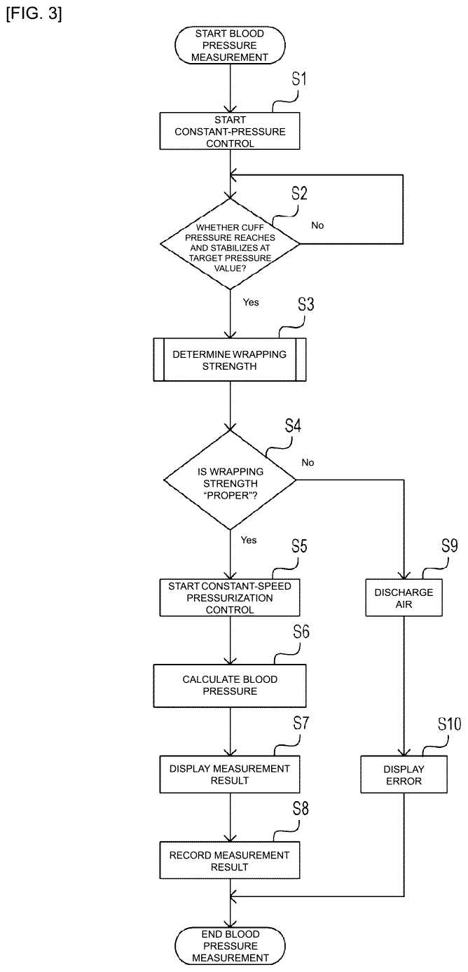

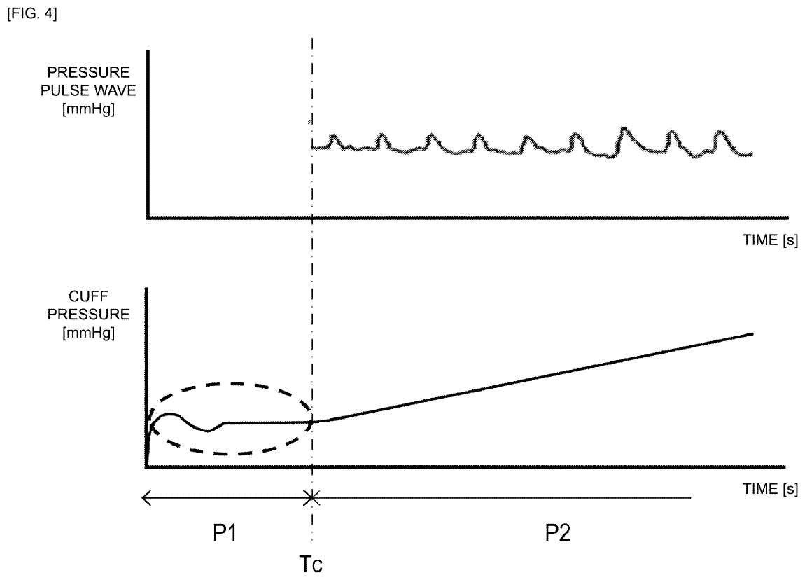

FIG. 3 is a flowchart showing an entire processing procedure of a blood pressure measurement method that is executed by the blood pressure measurement device 1. The entire processing for blood pressure measurement shown in FIG. 3 is stored in advance as a program in a predetermined area of the memory 24, and is implemented when the CPU 100 reads and executes the program from the memory 24. The program may also be recorded in a computer-readable storage medium and read from the storage medium into the blood pressure measurement device 1. FIG. 4 is a graph illustrating temporal changes in a pressure pulse wave and a cuff pressure detected during blood pressure measurement, both shown on the same time axis.

When measuring blood pressure, the cuff 11 is wrapped in advance around a measurement target site such as an upper arm or a wrist of a subject. In addition, the description will be made assuming that the subject performs a predetermined setting by the operation switch 26 and instructs the device to start blood pressure measurement. Note that, in the blood pressure measurement device 1 that has received the instruction to start blood pressure measurement, predetermined initialization such as opening the exhaust valve 14 and setting the cuff pressure to the atmospheric pressure (initial pressure) is executed.

When blood pressure measurement is started, the pressure control unit 120 starts constant-pressure control (step S1). At this time, the pressure control unit 120 controls the pump drive circuit 22 so as to maintain the cuff pressure detected by the pressure sensor 12 constant (referred to as “constant-pressure control”), using an initial pressurization end pressure value, which is a cuff pressure at which the initial pressurization ends, as a target pressure value. The constant-pressure control may be performed by, for example, PID control, but the control method is not limited thereto.

The condition determination unit 140 monitors the cuff pressure detected by the pressure sensor 12, and determines whether the cuff pressure reaches and stabilizes at the target pressure value (step S2). Whether the cuff pressure stabilizes may be determined, for example, based on whether a deviation of the cuff pressure from the target pressure value is within a range of ±5%, but the determination criterion is not limited thereto.

When it is determined in step S2 that the cuff pressure does not reach or stabilize at the target pressure value, step S2 is repeated, and the condition determination unit 140 continues monitoring the cuff pressure. Before proceeding to step S3, the sampled cuff pressure is stored in a predetermined area of the memory 24 in association with sampling time information.

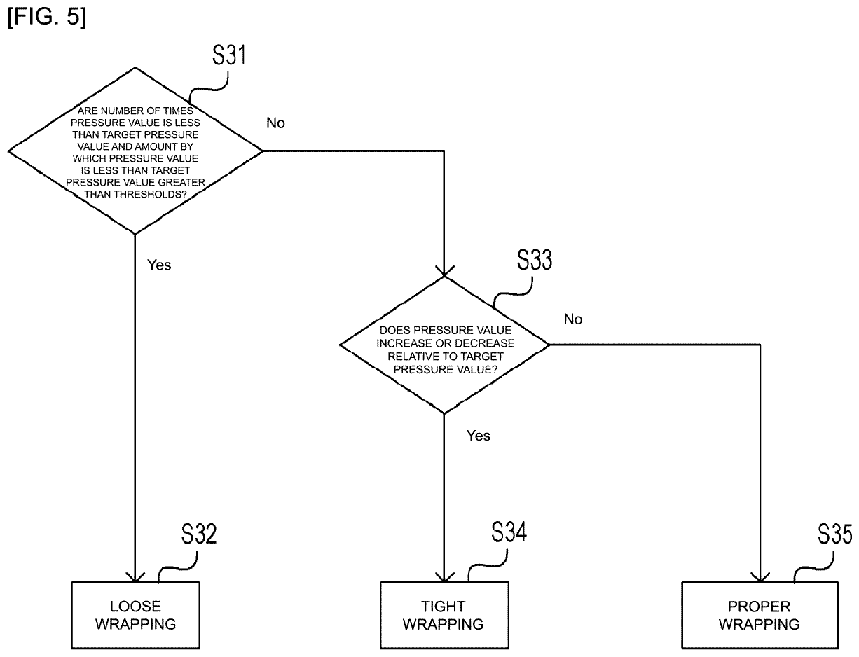

When the condition determination unit 140 determines in step S2 that the cuff pressure reaches and stabilizes at the target pressure value, the wrapping strength determination unit 150 executes wrapping strength determination processing shown in FIG. 5, based on the information on the temporal change in the cuff pressure stored in the memory 24 (step S3). Thus, the wrapping strength determination processing is performed under the constant-pressure control.

In the wrapping strength determination processing in step S3, the wrapping strength determination unit 150 determines the wrapping strength, based on a variation pattern of the cuff pressure during a period from an initial pressure until the cuff pressure reaches and stabilizes at the target pressure value under the constant-pressure control. Here, it is determined which of three categories, loose wrapping, proper wrapping, and tight wrapping, the wrapping strength of the cuff 11 falls into. When the cuff 11 is wrapped around a measurement target site such as an upper arm to form a cylindrical shape, and when a circumferential length of the cylinder is substantially equal to a circumference of the measurement target site, a pressure applied to the measurement target site by the cuff 11 becomes an appropriate level for blood pressure measurement, and therefore such a state is referred to as proper wrapping. When the circumferential length of the cylinder formed by the cuff 11 is shorter than the circumference of the measurement target site, the cuff 11 is tightly wrapped around the measurement target site, and a pressure applied to the measurement target site by the cuff 11 is higher than the appropriate level, and therefore such a state is referred to as tight wrapping. When the circumferential length of the cylinder formed by the cuff 11 is longer than the circumference of the measurement target site, the cuff 11 is loosely wrapped around the measurement target site, and a pressure applied to the measurement target site by the cuff 11 is lower than the appropriate level, and therefore such a state is referred to as loose wrapping. Here, the period from the initial pressure until the cuff pressure reaches and stabilizes at the target pressure value (in FIG. 4, the period P1 until Tc) during the constant-pressure control corresponds to a determination target period of the present invention. The time Tc corresponds to the end of the wrapping strength determination processing.

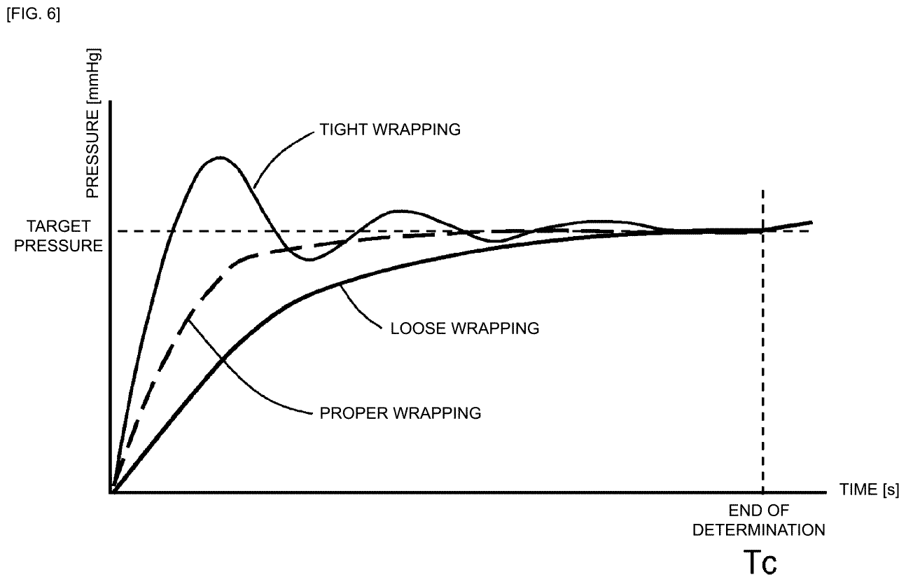

FIG. 6 is a graph illustrating an example of variation patterns of the cuff pressure when the wrapping strength is determined to be loose wrapping, proper wrapping, and tight wrapping. FIG. 6 is an enlarged view showing a portion surrounded by the broken line, which indicates the change in the cuff pressure in FIG. 4. In the loose wrapping state, a so-called undershoot state in which the cuff pressure rises slowly during pressurization and remains significantly below the target pressure value continues before reaching the target pressure value. In the tight wrapping state, the cuff pressure sharply rises immediately after the start of pressurization and exceeds the target pressure value, that is, enters a so-called overshoot state. Subsequently, the cuff pressure repeatedly increases and decreases, and reaches the target pressure value while vibrating above and below the target pressure value. In the proper wrapping state, the cuff pressure rises with pressurization but does not exceed the target pressure value, and reaches the target pressure value after quickly approaching the target pressure value.

The wrapping strength determination unit 150 determines whether the number of times and an amount by which the cuff pressure is less than the target pressure value from the initial pressure until the cuff pressure reaches the target pressure value are greater than predetermined thresholds (step S31). Here, the amount less than the target pressure value refers to a magnitude of a difference between the target pressure value and the cuff pressure.

When it is determined in step S31 that the number of times and the amount by which the cuff pressure is less than the target pressure value until the cuff pressure reaches the target pressure value are greater than the predetermined thresholds, it is determined that the wrapping strength is loose wrapping (step S32).

When it is determined in step S31 that the number of times and the amount by which the cuff pressure is less than the target pressure value until the cuff pressure reaches the target pressure value are equal to or less than the predetermined thresholds, the wrapping strength determination unit 150 determines whether the pressure value increases or decreases with respect to the target pressure value (step S33).

When it is determined in step S33 that the pressure value increases or decreases relative to the target pressure value until the cuff pressure reaches the target pressure value, it is determined that the wrapping strength is tight wrapping (step S34).

When it is determined in step S33 that the cuff pressure does not increase or decrease relative to the target pressure value until the cuff pressure reaches the target pressure value, it is determined that the wrapping strength is proper wrapping (step S35).

When it is determined to be proper wrapping in the wrapping strength determination in step S3, the initial pressurization by the constant-pressure control is ended, and the pressure control unit 120 starts to control the pump drive circuit 22 so that the cuff pressure increases at a constant speed (referred to as “constant-speed pressurization control”) (step S5). The constant-speed pressurization control is executed during a period P2 after Tc in FIG. 4.

The initial pressurization end pressure value (target pressure value) is set to a pressure lower than 30 mmHg, which is a typical pressure during a constant-voltage drive of the related art, for example, to about 10 mmHg. By setting the initial pressurization end pressure value to a lower level in this manner, pressure pulse waves for blood pressure measurement can be acquired from a lower pressure after an end of the wrapping strength determination processing.

In addition, at the end of the wrapping determination strength determination processing in step S3, a pump voltage (a drive voltage of the pump drive circuit 22) in an equilibrium state during the constant-pressure control is maintained, and the control is switched from that state to the constant-speed pressurization control to start acquiring pressure pulse waves for blood pressure measurement. This prevents a sudden change in the pressurization speed, thereby suppressing the influence of noise at the timing of control switching and enabling acquisition of clear changes in the pressure pulse wave.

The blood pressure calculation unit 130 performs blood pressure calculation processing in which, under constant-speed pressurization control, a pressure pulse wave for blood pressure measurement is acquired from the HPF unit of the pressure detection unit 110, and a blood pressure value such as a systolic blood pressure and a diastolic blood pressure is calculated by an oscillometric method (step S6).

The CPU 100 displays the measurement result such as the blood pressure value calculated in step S6 on the display unit 25 of the blood pressure measurement device 1 (step S7). Then, the CPU 100 records the measurement result such as the blood pressure value calculated in step S6 in a predetermined area of the memory 24 of the blood pressure measurement device 1 (step S8), and ends the blood pressure measurement processing.

When it is determined in the wrapping strength determination processing in step S3 that the wrapping strength is not proper, that is, when it is determined to be loose wrapping or tight wrapping, the pressure control unit 120 controls the valve drive circuit 23 to open the exhaust valve 14, thereby discharging air from the air bag 11a of the cuff 11 (step S9). Then, the CPU 100 causes the display unit 25 to display an error (step S10), and ends the blood pressure measurement processing.

### Example 2

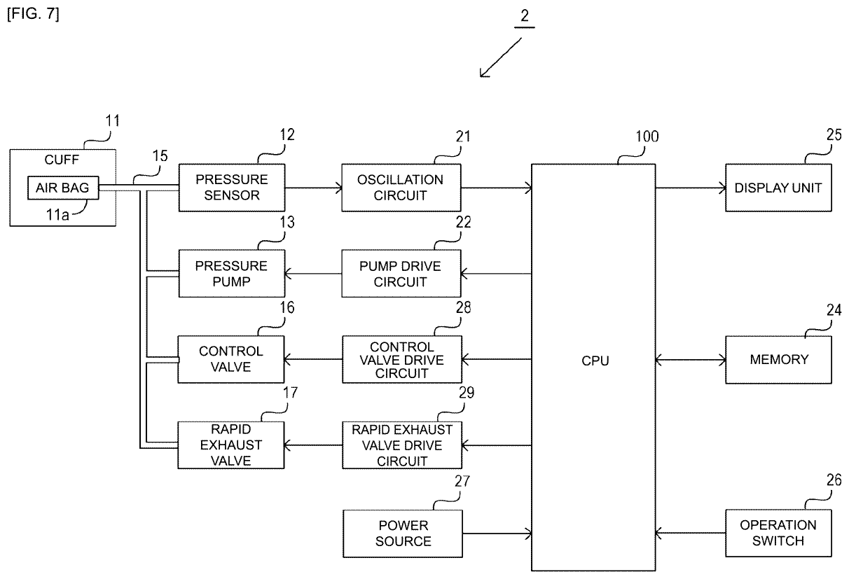

FIG. 7 is a block diagram schematically illustrating a hardware configuration of a blood pressure measurement device 2 according to Example 2. Configurations common to those of the blood pressure measurement device 1 according to Example 1 are denoted by the same reference numerals, and detailed description thereof will be omitted.

The blood pressure measurement device 2 includes a control valve 16 and a control valve drive circuit 28 instead of the exhaust valve 14 and the valve drive circuit 23, and further includes a rapid exhaust valve 17 and a rapid exhaust valve drive circuit 30. The blood pressure measurement device 2 corresponds to a blood pressure measurement device of the present invention. In addition, the control valve 16 corresponds to an exhaust valve of the present invention.

The control valve 16 is a valve whose opening degree can be continuously controlled, and the control valve drive circuit 28 controls the opening degree of the control valve 16 by a control signal output from the CPU 100. The rapid exhaust valve 17 is a valve that can rapidly exhaust air by opening the valve and immediately reduce the cuff pressure. The rapid exhaust valve drive circuit 30 controls the opening and closing of the rapid exhaust valve 17 by a control signal output from the CPU 100. In the blood pressure measurement device 2, the pressure control unit 120 controls the operations of the pump drive circuit 22, the control valve drive circuit 28, and the rapid exhaust valve drive circuit 29.

Since it is difficult to delicately control the cuff pressure with the pressurization pump 13, the constant-pressure control at a low pressure can be achieved in a shorter time by combining the control valve 16 capable of slow exhaust. The control valve 16 is controlled to open and close during the constant-pressure control, but the control valve 16 is closed during the constant-speed pressurization control.

The overall processing procedure for blood pressure measurement including the wrapping strength determination processing is the same as that of the blood pressure measurement device 1 according to Example 1, and thus the description thereof will be omitted.

In addition, the blood pressure measurement device 2 includes the rapid exhaust valve 17, which allows the cuff pressure to be lowered in a short time during exhaust, thereby reducing the burden on the subject.

Thusly, the embodiments shown and described are merely exemplary and various alternatives, combinations, omissions, of specific components, or foreseeable alternative components, understood by one having ordinary skill in the art, described in the present invention or within the field of the present disclosure, are intended to fall within the scope of the appending claims.

It will be appreciated that various aspects of the present invention and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the claims.

### REFERENCE SIGNS LIST

1, 2 . . . Blood pressure measurement device
11 . . . Cuff
110 . . . Pressure detection unit
120 . . . Pressure control unit
130 . . . Blood pressure calculation unit
150 . . . Wrapping strength determination unit

What is claimed is:

1. A blood pressure measurement device comprising:
a cuff to be wrapped around a measurement target site;
a pressure detection unit configured to detect a cuff pressure in the cuff;
a pressure control unit configured to control the cuff pressure;
a blood pressure calculation unit configured to perform blood pressure calculation processing to calculate a blood pressure of a subject on a basis of the cuff pressure detected; and,
a wrapping strength determination unit configured to perform wrapping strength determination processing to determine a wrapping strength of the cuff to the measurement target site, wherein:
the pressure control unit performs a constant-pressure control to maintain the cuff pressure at a constant target pressure value from a start of an increase in the cuff pressure to an end of the wrapping strength determination processing, and performs a constant-speed pressurization control to increase the cuff pressure at a constant speed for calculating the blood pressure after the end of the wrapping strength determination processing; and,
the wrapping strength determination unit determines the wrapping strength during the constant-pressure control.

2. The blood pressure measurement device according to claim 1, wherein the wrapping strength determination unit determines the wrapping strength on a basis of a change in the cuff pressure during a determination target period during which the cuff pressure reaches the target pressure value from an initial pressure, and stabilizes.

3. The blood pressure measurement device according to claim 2, wherein the wrapping strength determination unit determines the wrapping strength by comparing the number of times the cuff pressure becomes less than the target pressure value during the determination target period with a predetermined threshold, and comparing a difference from the target pressure value with a predetermined threshold.

4. The blood pressure measurement device according to claim 2, wherein the wrapping strength determination unit determines the wrapping strength on a basis of whether the cuff pressure increases or decreases relative to the target pressure value during the determination target period.

5. The blood pressure measurement device according to claim 3, wherein the wrapping strength determination unit determines the wrapping strength on a basis of whether the cuff pressure increases or decreases relative to the target pressure value during the determination target period.

6. The blood pressure measurement device according to claim 1, wherein:
the target pressure value is smaller than 30 mmHg; or,
the wrapping strength determination unit determines the wrapping strength on a basis of a change in the cuff pressure during a determination target period during which the cuff pressure reaches the target pressure value from an initial pressure, and stabilizes, and further, the target pressure value is smaller than 30 mmHg.

7. The blood pressure measurement device according to claim 1, wherein:
the pressure control unit performs the constant-speed pressurization control depending on a result of the wrapping strength determination processing; or,
the wrapping strength determination unit determines the wrapping strength on a basis of a change in the cuff pressure during a determination target period during which the cuff pressure reaches the target pressure value from an initial pressure, and stabilizes, and further, the pressure control unit performs the constant-speed pressurization control depending on a result of the wrapping strength determination processing.

8. The blood pressure measurement device according to claim 1, wherein:
the pressure control unit performs the constant-pressure control by a PID control; or,
the wrapping strength determination unit determines the wrapping strength on a basis of a change in the cuff pressure during a determination target period during which the cuff pressure reaches the target pressure value from an initial pressure, and stabilizes, and further, the pressure control unit performs the constant-pressure control by a PID control.

9. The blood pressure measurement device according to claim 1, wherein:
the pressure control unit controls a pump configured to supply air to the cuff, and an exhaust valve configured to control exhaust from the cuff; or,
the wrapping strength determination unit determines the wrapping strength on a basis of a change in the cuff pressure during a determination target period during which the cuff pressure reaches the target pressure value from an initial pressure, and stabilizes, and further, the pressure control unit controls a pump configured to supply air to the cuff, and an exhaust valve configured to control exhaust from the cuff.

10. A blood pressure measurement method comprising:
detecting a cuff pressure in a cuff wrapped around a measurement target site;
performing a constant-pressure control to maintain the cuff pressure at a constant target pressure value;
performing wrapping strength determination processing to determine a wrapping strength of the cuff to the measurement target site during the constant-pressure control;
performing a constant-speed pressurization control to increase the cuff pressure at a constant speed after an end of the wrapping strength determination processing; and,
performing blood pressure calculation processing to calculate a blood pressure of a subject on a basis of the cuff pressure during the constant-speed pressurization control.

11. A blood pressure measurement recording medium configured to cause a computer to execute:
detecting a cuff pressure in a cuff wrapped around a measurement target site;
performing a constant-pressure control to maintain the cuff pressure at a constant target pressure value;
performing wrapping strength determination processing to determine a wrapping strength of the cuff to the measurement target site during the constant-pressure control;
performing a constant-speed pressurization control to increase the cuff pressure at a constant speed after an end of the wrapping strength determination processing; and,
performing blood pressure calculation processing to calculate a blood pressure of a subject on a basis of the cuff pressure during the constant-speed pressurization control.
